(19) United States
(12) Patent Application Publication
(10) Pub. No.: US 2025/0169740 A1
(43) Pub. Date: May 29, 2025

(54) SYSTEMS AND METHODS FOR GUIDED EEG STIMULATION

(71) Applicant: Ice Neurosystems, Inc., Washington, DC (US)

(72) Inventor: Allen Edward WAZIRI, Washington, DC (US)

(73) Assignee: Ice Neurosystems, Inc., Washington, DC (US)

(21) Appl. No.: 18/844,264

(22) PCT Filed: Feb. 1, 2023
(86) PCT No.: PCT/US2023/061730

§ 371 Date: Sep. 5, 2024

Related U.S. Application Data
(60) Provisional application No. 63/269,125, filed on Mar. 10, 2022.

(51) Int. Cl. A61B 5/383; G16H 10/60; G16H 20/10; G16H 50/20; G16H 50/70

(52) U.S. Cl. CPC A61B 5/383; G16H 10/60; G16H 20/10; G16H 50/20; G16H 50/70

(57) ABSTRACT

A method for generating a reactivity profile of a patient includes: causing a reactivity device to generate a plurality of reactivity outputs according to a predetermined sequence, the plurality of reactivity outputs respectively associated with different stimulus modalities; in parallel with the predetermined sequence, receiving stimulus data that includes electroencephalogram (EEG) data captured by sensors applied to the patient; and forming the reactivity profile by temporally aligning the received stimulus data with the predetermined sequence, the aligning including defining segments of the received stimulus data based on temporal positions of the plurality of reactivity outputs in the predetermined sequence.

CROSS-REFERENCE TO RELATED APPLICATION(S)

This is a National Stage Application under 35 U.S.C. § 371 of International Application No. PCT/US2023/061730, filed on Feb. 1, 2023, which claims priority to U.S. Provisional Application No. 63/269,125, filed on Mar. 10, 2022, the entireties of which are incorporated herein by reference.

### TECHNICAL FIELD

Various embodiments of this disclosure relate generally to techniques for guided Electroencephalography (EEG) stimulation, and, more particularly, to systems and methods for generation and/or standardization of a reactivity profile of a patient.

### BACKGROUND

Current techniques of performing EEG reactivity testing generally involve a physician, such as a neurologist, physically administering an EEG reactivity test at a patient's bedside. Such EEG reactivity testing is customarily subjective, with no standard for the type of stimulus or the length of stimulus when performing the EEG reactivity test. This may result in a subjective performance of the EEG reactivity test, as well as subjective documentation of the corresponding EEG reactivity test results. Such subjective EEG reactivity test results may be difficult or impossible to generalize across the same patient, let alone across different, patients.

Further, conventional techniques, including the foregoing, fail to provide a technique for standardizing the EEG reactivity test administration process. Conventional techniques also fail to provide a technique for standardizing the EEG reactivity test data collection process. This failure may result in a decreased accuracy of EEG reactivity test data, which may negatively impact patient diagnosis, treatment, and/or care.

This disclosure is directed to addressing one or more issues such as the above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for generating a reactivity profile of a patient.

In one aspect, an exemplary embodiment of a method for generating a reactivity profile of a patient may include: causing, via at least one processor of a reactivity system, a reactivity device to generate a plurality of reactivity outputs according to a predetermined sequence, the plurality of reactivity outputs respectively associated with different stimulus modalities. The method may further include: in parallel with the predetermined sequence, receiving, via the at least one processor of the reactivity system, stimulus data that includes electroencephalogram (EEG) data captured by sensors applied to the patient. The method may further include: forming the reactivity profile by, via the at least one processor of the reactivity system, temporally aligning the received stimulus data with the predetermined sequence, the aligning including defining segments of the received stimulus data based on temporal positions of the plurality of reactivity outputs in the predetermined sequence.

In a further aspect, an exemplary embodiment of a computer system for generating a reactivity profile of a patient may include: a reactivity device operable to generate a reactivity output, a plurality of sensors configured to be applied to the patient, at least one memory storing processor-readable instructions, and at least one processor operatively connected to the reactivity device, the plurality of sensors, and the at least one memory, where the at least one processor is configured to execute the processor-readable instructions to perform a plurality of operations. The operations may include: causing the reactivity device to generate a plurality of reactivity outputs according to a predetermined sequence, the plurality of reactivity outputs respectively associated with different stimulus modalities, in parallel with the predetermined sequence, receiving, via the reactivity device, stimulus data that includes electroencephalogram (EEG) data captured by the plurality of sensors, and forming the reactivity profile by temporally aligning the received stimulus data with the predetermined sequence, the aligning including defining segments of the received stimulus data based on temporal positions of the plurality of reactivity outputs in the predetermined sequence.

In a further aspect, an exemplary embodiment of a non-transitory computer-readable medium comprising instructions that are executable by a processor to perform operations for generating a reactivity profile of a patient, may include operations for: causing, via a reactivity system, a reactivity device to generate a plurality of reactivity outputs according to a predetermined sequence, the plurality of reactivity outputs respectively associated with different stimulus modalities, in parallel with the predetermined sequence, receiving, via the reactivity system, stimulus data that includes electroencephalogram (EEG) data captured by sensors applied to a patient, and forming the reactivity profile by, via the reactivity system, temporally aligning the received stimulus data with the predetermined sequence, the aligning including defining segments of the received stimulus data based on temporal positions of the plurality of reactivity outputs in the predetermined sequence.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1A depicts an exemplary environment that may be utilized with techniques presented herein, according to one or more embodiments.

FIG. 1B depicts an exemplary system that may be utilized with techniques presented herein, according to one or more embodiments.

FIG. 2 depicts an exemplary process for generating a reactivity profile of a patient, according to one or more embodiments.

FIG. 3 depicts an exemplary method for displaying a reactivity profile of a patient, according to one or more embodiments

FIG. 4 depicts an exemplary method for generating a portal for providing access to a library of reactivity profiles of a patient, according to one or more embodiments.

FIG. 5 depicts a “Prepare for Reactivity Test” page of an exemplary graphical user interface, according to one or more embodiments.

FIG. 6 depicts a “Stimulation Parameters” page of an exemplary graphical user interface, according to one or more embodiments.

FIG. 7 depicts a “Starting Test” page of an exemplary graphical user interface, according to one or more embodiments.

FIG. 8 depicts a “Stimulate” page of an exemplary graphical user interface, according to one or more embodiments.

FIG. 9 depicts a “Stop Stimulation” page of an exemplary graphical user interface, according to one or more embodiments.

FIG. 10 depicts a “Test Complete” page of an exemplary graphical user interface, according to one or more embodiments.

FIG. 11 depicts a reactivity profile interface of aligned received stimulus-associated raw EEG waveform data of an exemplary graphical user interface, according to one or more embodiments.

FIG. 12 depicts a main reactivity profile interface of the aligned received stimulus-associated processed EEG data and received sensor data of an exemplary graphical user interface, according to one or more embodiments.

FIG. 13 depicts an overview interface of an exemplary graphical user interface, according to one or more embodiments.

FIG. 14 depicts a simplified functional block diagram of a computer that may be configured as a device for executing the methods of the disclosure, according to one or more embodiments.

### DETAILED DESCRIPTION OF EMBODIMENTS

According to certain aspects of the disclosure, methods and systems are disclosed for the standardization of EEG reactivity testing data collection. Physically administering an EEG reactivity test and interpreting the EEG reactivity test data has customarily been a very subjective process. Conventional techniques generally fail to provide a standardized methodology for administering the EEG reactivity test. Additionally, since the EEG reactivity test administration process is not standardized, the EEG data collection process is not standardized either. For example, conventional techniques do not provide a standard type of stimulus or length of stimulus when performing the EEG reactivity test. Such lack of standardization may make it difficult or impossible to compare different test results for the same patient, let alone across different patients. Accordingly, improvements in technology relating to the standardization of EEG reactivity test administration and data collection are needed.

In comatose or otherwise profoundly neurologically impaired patients, standard approaches to neurological testing that rely on physical examination generally cannot provide insight into the severity of a brain injury. Coma may be a reversible process that will improve with time and treatment. However, in some cases, the brain may be so profoundly damaged that there is little to no hope for a meaningful recovery. For example, a patient who suffered from cardiac arrest and underwent subsequent cardiopulmonary resuscitation may have an irreversible amount of injury to the brain, e.g., due to a period of loss of blood flow to the brain. However, in other cases, the blood flow to the brain may have been restored in time to avoid permanent injury.

In both of the previously described cases, the patient may reside in a comatose state for days to weeks following the cardiac arrest, resulting in an uncertainty of outcome. From a clinical perspective, this may result in the patient receiving prolonged intensive care that is otherwise futile given irreversible brain injury. Alternatively, decisions may be made to withdraw supportive care for a patient who may otherwise have a chance for recovery and would otherwise warrant continued aggressive clinical measures.

Electroencephalography (EEG) reactivity testing can be a critical means of providing concrete data supporting clinical decision making in cases of profoundly neurologically impaired patients. For example, when the EEG is nonreactive there is significant concern for widespread, devastating brain injury. Additionally, when the EEG is reactive there is evidence that the brain has the capacity to receive and respond to external stimuli, suggesting maintenance of at least some functional substrate.

EEG is a technique through which electrical signals, generated as a natural function of neuronal firing in the brain during periods of health and disease, are recorded by platforms which generally include components such as electrodes, hardware amplification equipment, and bedside computers running specialized software programs. The hardware of an EEG system may include one or more electrodes such as scalp electrodes and a bedside computer system. A user may place the scalp electrodes on a patient's scalp to record the EEG. The bedside computer system may collect and display EEG data. The user may review real-time EEG data associated directly on the computer screen at the bedside. In some instances, EEG data may be off-loaded or recorded for later viewing. However, such a system generally has little or no capacity for live transmission, which may limit the ability for a remote user to review the time-locked data at a distance.

The detection of changes in EEG patterns, which are related to external stimuli, can be useful for determining the extent of a brain injury, as well as the likelihood of a neurological recovery. Such external stimuli may be considered as afferent inputs through the nervous system that are “detected” by the brain and result in corresponding changes in the firing patterns of brain neurons.

Changes in EEG patterns may by documented by EEG reactivity testing, which is a clinical intervention. Conventional approaches to EEG reactivity testing require a clinician to perform one or more physical stimulations to the patient, such as noxious tactile or verbal stimulation. Simultaneously to performing the one or more physical stimulations to the patient, the clinician reviews an EEG recording to determine whether baseline EEG patterns change in relationship to the stimulus onset and offset. Such changes are generally interpreted in subjective fashion by the clinician performing the test, and are subsequently documented to be “reactive” or “nonreactive.”

Currently, conventional methods of performing EEG reactivity testing are not standardized. For example, there is no standard for type of stimulus, length of stimulus, need for performance of multiple stimulation trials with intervening “rest” periods, or specification of changes in the EEG data that constitute a “reactive” versus “nonreactive” EEG. Furthermore, when performing EEG reactivity testing, clinical event annotation cannot be reliably time-locked to the corresponding EEG reactivity testing data. In other words, the simultaneous performance of stimulation and review of the EEG is customarily required of an in-person clinician because it may be difficult or impossible to temporally align the EEG test results with a timeline of the performance of stimulus after the fact, and even a slight temporal misalignment may negatively impact the accuracy or the EEG test. This reality makes post-hoc association of potential changes in the EEG reactivity testing data record with clinical action or physiological events difficult, particularly such physiological events that occur on the order of seconds, which is expected to be seen when performing EEG reactivity testing.

Moreover, interpretation of the EEG reactivity testing data is customarily subjective and physician dependent as well. For example, there are no standardized means by which neurologists document the results of EEG reactivity testing. The conventional method of notating EEG reactivity testing results involves notations that are made separately in a patient's medical record. The reliability of human notations on a time-specific basis in the EEG record may be inaccurate, due to human error or neurological events that do not appear to be important to the patient and/or clinician. As a result, the notations are unable to be appreciated when a physician, such as a neurologist, reviews the EEG data in isolation. Therefore, performance and documentation of EEG reactivity testing by the neurologist is necessarily subjective and the EEG reactivity test data are impossible to generalize across patients. Additionally, in cases that involve the same patient, there are still subjectivity issues when different neurologists perform the testing for the same patient.

Reference to any particular activity is provided in this disclosure only for convenience and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

In an exemplary use case, a computer-assisted system for performing a standardized EEG reactivity test includes software-driven commands regarding, for example, one or more of stimulus modalities, specificity of timing for stimulation periods and washout periods, repetition of stimulation trials, etc. Such commands may be given to a bedside nurse and/or through bedside associated devices. The bedside nurse may receive the commands through a system display. The bedside associated devices may provide stimulation by delivering focal noxious electrical stimulus, loud auditory stimulus, photic stimulation, etc. Such bedside associated devices may provide further EEG reactivity testing standardization and remove the need for a bedside nurse. Either way, the system may remove the need for the physical presence of a physician, such as a neurologist, at the patient's bedside, to perform the physical manipulations associated with EEG reactivity testing. Removing the need for the physician to administer the EEG reactivity test may allow for the physician to review the EEG data from a distance.

The standardization of data collection may provide a more accurate and/or physiologically relevant interpretation of EEG data associated with reactivity testing. The standardization of EEG data collection may include, for example, documenting clinical variables and/or other physiological variables. For instance, such clinical variables may include medications. Additionally, for example, such physiological variables may include the presence of neurological responses to stimulus, and specific, time-locked annotation of stimulus onset and offset periods. Such documentation may occur through concurrent extraction of relevant medication data from the electronic medical record and time-locked collection of systemic physiological data from bedside monitors.

Such a system also may allow for the gathering of robust data that may strengthen capabilities for post-hoc data review and machine learning analyses. Standardization of data collection may also allow for the development of algorithms to automate EEG data analysis through supervised machine learning. For example, the development of EEG data interpretation algorithms may allow a machine to interpret the EEG data, and/or remove the need for post-hoc interpretation by a trained specialist.

The system may be configured to provide quality control to confirm that the EEG system correctly collects the EEG data, and/or confirm that the stimuli were being applied effectively. For example, impedance data from the EEG electrodes may be evaluated in real time to confirm the validity of electrical signals from a bioelectronic standpoint during test performance. Additional EEG artifact detection and removal algorithms may be applied to avoid the potential for spurious data to influence interpretation of EEG reactivity analysis. Additionally, for example, microphones may be used to detect the amplitude of verbal stimulation made by the bedside nurse and/or bedside associated devices. Furthermore, motion detectors or cameras may be used to detect actions associated with physical stimulation made by the bedside nurse and/or bedside associated devices.

It should be understood that the examples above are illustrative only. The techniques and technologies of this disclosure may be adapted to any suitable activity.

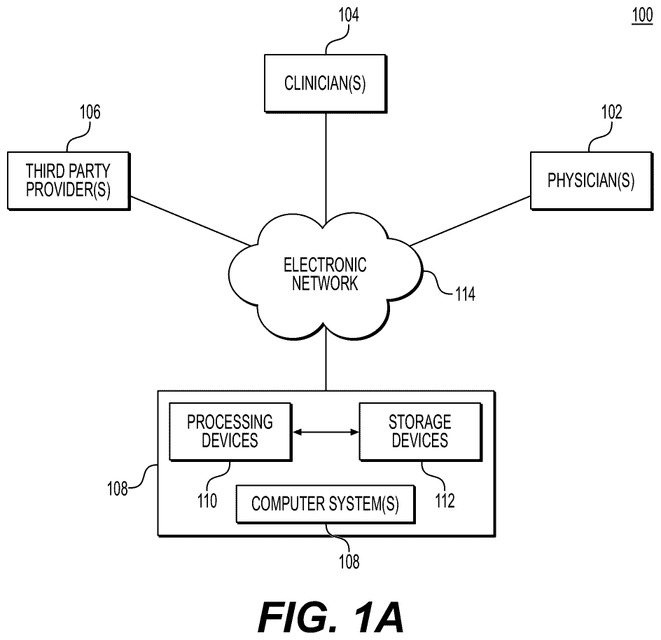

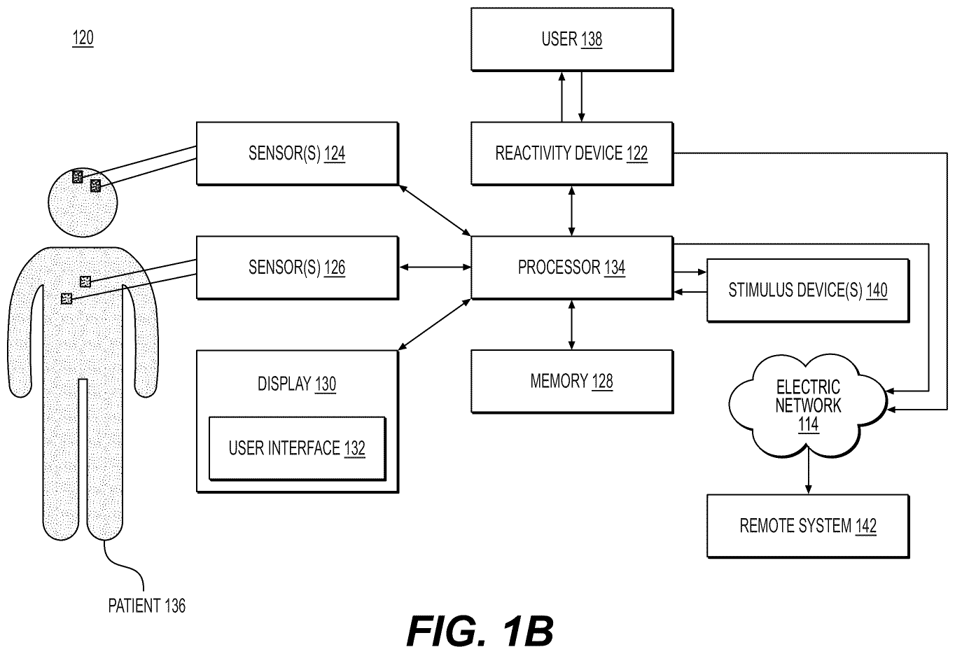

FIG. 1A depicts an exemplary environment 100 that may be utilized with techniques presented herein, according to one or more embodiments. Specifically, FIG. 1A depicts a plurality of physicians 102, clinicians 104, and third party providers 106, any of whom may be connected to an electronic network 114, such as the Internet, through one or more computers, servers, and/or handheld mobile devices. As will be discussed in further detail below, one or more computer system(s) 108, e.g., for performing activities such as performing a reactivity test and/or generating or standardizing a reactivity profile, may communicate and/or be accessed by other components in the environment 100.

The physicians 102 may include, for example, medical doctors, neurologists, Emergency Room doctors, etc. The clinicians 104 may include, for example, nurses, nurse practitioners, etc. The third party providers 106 may include, for example, contractors, vendors, etc.

In various embodiments, the electronic network 114 may be a wide area network (“WAN”), a local area network (“LAN”), personal area network (“PAN”), or the like. In some embodiments, electronic network 100 includes the Internet, and information and data provided between various systems occurs online.

In some embodiments, the components of the environment 100 are associated with a common entity, e.g., a hospital, facility, or the like. In some embodiments, one or more of the components of the environment is associated with a different entity than another. The systems and devices of the environment 100 may communicate in any arrangement.

Physicians 102, clinicians 104, and/or third party providers 106 may create or otherwise obtain a patient reactivity profile by performing an EEG reactivity test. The physicians 102, clinicians 104, and/or third party providers 106 may also obtain any combination of patient-specific information, such as age, medical history, blood pressure, blood viscosity, etc.

Physicians 102, clinicians 104, and/or third party providers 106 may transmit the reactivity profile and/or patient-specific information to computer system(s) 108 over the electronic network 114. Computer system(s) 108 may include an electronic medical data system, computer-readable memory such as a hard drive, flash drive, disk, etc., and/or other components as discussed in further detail below.

Computer system(s) 108 may include processing devices 110 and storage devices 112. The processing devices 110 may process data stored in the storage devices. The storage devices 112 may store data received from physicians 102, clinicians 104, and/or third party providers 106. In some embodiments, the storage devices 112 include and/or interact with an application programming interface for exchanging data to other server systems, e.g., one or more of the other components of the environment. The storage devices 112 may include and/or act as a repository or source for EEG data and/or sensory data. Alternatively, or in addition, the present disclosure (or portions of the system and methods of the present disclosure) may be performed on a local processing device (e.g., a laptop), absent an external server or network.

Although depicted as separate components in FIG. 1A, it should be understood that a component or portion of a component in the environment 100 may, in some embodiments, be integrated with or incorporated into one or more other components. In some embodiments, operations or aspects of one or more of the components discussed above may be distributed amongst one or more other components. Any suitable arrangement and/or integration of the various systems and devices of the environment 100 may be used.

FIG. 1B depicts an exemplary embodiment of a system that may be utilized with techniques presented herein, e.g., as or with the computer system(s) 108 of FIG. 1A, according to one or more embodiments. Specifically, FIG. 1B depicts a system 120 that may include a patient 136, reactivity device 122, a plurality of sensors 124, 126, at least one memory 128, a display 130, at least one processor 134, a user 138, a stimulus device(s) 140, a remote system 142, and an electric network 114.

The reactivity device 122 may be operable to generate a reactivity output, and may be operatively connected to the plurality of sensors 124, 126, to the display 130, and/or to a remote system 142.

In some embodiments, the reactivity output may include at least one instruction for a user 138 (e.g., physicians 102, clinicians 104, and/or third party providers 106) to perform a stimulus, e.g., with or without the aid of a user-operated stimulus device 140. Illustrative examples of such instructions include an instruction to perform a sternal rub, say the patient's name, clap, pinch the patient, operate a shock device, use smelling salts, etc. Any suitable instruction for any suitable stimulus application by a user 138 may be used.

In some embodiments, e.g., instead of or in combination with stimulus instructions for a user 138, the reactivity output may include at least one instruction for a stimulus device(s) 140 that is operable to automatically generate a stimulus. Example stimulus device(s) 140 may include an audio device such as headphones, a visual device such as light-emitting goggles or a light device, a noxious device such as a shock device, etc. It should be understood that the aforementioned examples are illustrative only, and that any suitable stimulus device or combination of stimulus devices may be used.

The plurality of sensors 124, 126, may be configured to be applied to a patient 136. The plurality of sensors 124 may include any suitable type of EEG sensor (e.g., scalp electrodes) and may be configured to capture EEG data. The scalp electrodes may be connected, such as by a wired connection or a wireless connection, to an amplifier element and/or other components known to one of ordinary skill in the art (not show), and the resulting EEG data may be transferred to a computer hardware element, e.g., the memory 128. Additionally, the scalp electrodes may be configured to capture the stimulus data in real-time. For example, the scalp electrodes may capture the stimulus data as the user 138 and/or stimulus device 140 applies stimuli to the patient. The plurality of sensors 126 may include any suitable type of biological or physiological sensor and may be configured to capture a biological property and/or a physiological property of the patient.

The at least one memory 128 may further store processor-readable instructions. The display 130 may include a user interface 132 that may be configured to display a reactivity profile and/or a previously formed reactivity profile. The reactivity profile may indicate a patient's 136 real-time reactions to an EEG reactivity test, as discussed in further detail below. The user interface 132 may include interactive elements. The remote system 142 may allow for the user 138 and/or a user separate from user 138 (e.g., a remote user) to remotely view the reactivity profile. In one embodiment, the user 138 and/or a remote user may use the remote system 142 to access a portal to view the reactivity profile. The remote system 142 may communicate with the processor 134 or the reactivity device 112 via electric network 114 (described in relation to FIG. 1). Additionally, the at least one processor 130 may be operatively connected to the reactivity device 122, the plurality of sensors 124, 126, the at least one memory 128, the display 130, the stimulus device(s) 140, and/or the electric network 114.

Although depicted as separate components in FIG. 1B, it should be understood that a component or portion of a component in the system 120 may, in some embodiments, be integrated with or incorporated into one or more other components. In some embodiments, operations or aspects of one or more of the components discussed above may be distributed amongst one or more other components. Any suitable arrangement and/or integration of the various systems and devices of the environment 120 may be used.

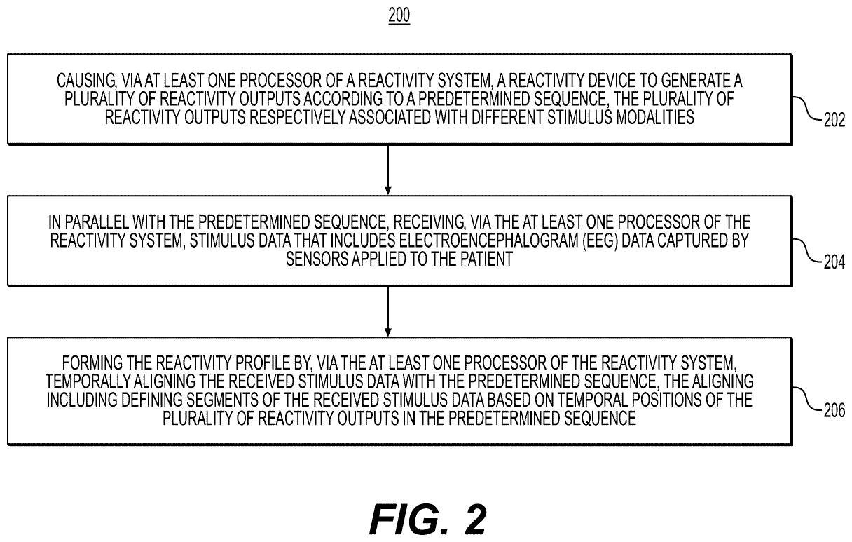

FIG. 2 depicts an exemplary process for generating a reactivity profile of a patient (e.g., patient 136), according to one or more embodiments.

The method may include applying sensors (e.g., sensors 124, 126) to the patient (e.g., patient 136), in order to receive stimulus data and/or sensor data from the patient. For example, scalp electrodes may be placed on a patient's scalp by the user. Such scalp electrodes may capture the patient's stimulus data (e.g., EEG data). Additionally, for example, physiological sensors (EKG leads, pressure monitors, oxygen sensors, etc.) may be applied to the patient (e.g., patient 136) to capture the patient's sensor data (e.g., heart rate, respiratory rate, arterial blood pressure, etc.).

The method may include causing, via at least one processor (e.g., processor 132) of a reactivity system (e.g., system 120), a reactivity device (e.g., reactivity device 122) to generate a plurality of reactivity outputs according to a predetermined sequence, the plurality of reactivity outputs respectively associated with different stimulus modalities (Step 202).

Each of the plurality of reactivity outputs may include at least one stimulus period and at least one washout period, where the at least one stimulus period may precede the at least one washout period. The at least one stimulus period may be a period of time where at least one stimulus modality is applied to the patient (e.g., patient 136). The at least one stimulus modality of the predetermined sequence may include at least one or more of, e.g., at least two of: at least one visual modality, at least one auditory modality, and/or at least one somatosensory modality, as discussed in further detail below. The at least one washout period may be a period of time where no stimulus is applied to the patient (e.g., patient 136), allowing for the patient's neurological functions to reset after the application of a stimulus modality. The at least one stimulus period may have a corresponding stimulus length based on a predetermined physiological stimulus period. The at least one stimulus period may correspond to the different stimulus modalities, where a stimulus modality is applied for the stimulus length to a patient (e.g., patient 136).

The predetermined physiological stimulus period may be a set period of time, which may be the same length for all of the different stimulus modalities, or the different lengths for each of the different stimulus modalities. For example, the predetermined physiological stimulus period may be 4 seconds for the visual modalities, the auditory modalities, and the somatosensory modalities. Additionally, for example, the predetermined physiological stimulus period may be 5 seconds for all visual modalities, 6 seconds for all auditory modalities, and 7 seconds for all somatosensory modalities. The predetermined physiological stimulus period may have been previously set by a test administrator. Additionally, in some embodiments, the predetermined physiological stimulus period may be changed/updated by a software/system update, system override, or user input. The predetermined physiological stimulus period may assist in standardizing the reactivity test, e.g., by making sure that the stimulus period is the same amount of time for all of the different stimulus modalities or for each of the different types of stimulus modalities. This standardization may be used to ensure, for example, that the different stimulus modalities are not applied for inconsistent periods of time.

The at least one washout period may have a length based on a predetermined post-stimulus settling period. Allowing the patient's neurological functions to reset assists in isolating the application of the stimulus modalities, resulting in a higher probability that the patient's reactions are responsive to the application of the present stimulus modality, and not an echo of the previous stimulus modality.

The predetermined post-stimulus settling period may be a standard period of time that the reactivity system implements after every stimulus modality application. The predetermined post-stimulus settling period may have been previously set by a test administrator. Additionally, the predetermined post-stimulus settling period may be changed/updated by a software/system update, system override, or user input. Including the predetermined post-stimulus period assists in standardizing the reactivity test because the washout period may be a set amount of time, allowing for equal washout periods between each stimulus modality.

As noted above, the plurality of reactivity outputs of the predetermined sequence may be respectively associated with different stimulus modalities. For example, the different stimulus modalities may include one or more of, e.g., at least two of: at least one visual modality, at least one auditory modality, and/or at least one somatosensory modality. The at least one visual modality may include, for example, forced eye opening of the patient (e.g., patient 136), shining light into the patient's eyes, and/or placing goggles on the patient that emit light/colors in a controlled fashion. The at least one auditory modality may include the user (e.g., user 138) clapping his or her hands, the user calling the patient's name, and/or placing headphones with machine-generated tones on the patient (e.g., patient 136). The at least one somatosensory modality may include sternal rubbing of the patient, applying nailbed pressure to the patient, pinching the patient (e.g., patient 136), and/or electrical impulse delivered by a device to the patient (e.g., patient 136). Other stimulus or combinations of stimulus may be used in various embodiments. Any suitable ordering of stimulus modalities for the predetermined sequence may be used.

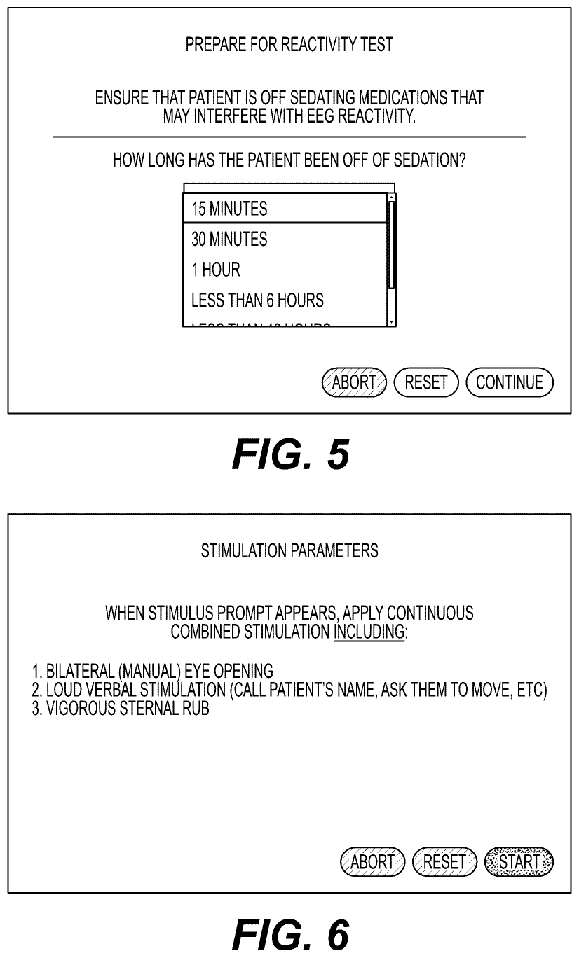

A user (e.g., user 138), a stimulus device (e.g., stimulus device 140), or both may apply the different stimulus modalities to a patient (e.g., patient 136). Additionally, the predetermined sequence of reactivity outputs generated and/or displayed on the reactivity system display (e.g., display 130) may be dependent upon whether the user (e.g., user 138) or the stimulus device (e.g., stimulus device(s) 140) are applying a stimulus modality. For example, if the stimulus device (e.g., stimulus device(s) 140) applies the stimulus modality to the patient (e.g., patient 136), the reactivity system display (e.g., display 130) may display a blank screen or a screen that states that the stimulus device (e.g., stimulus device(s) 140) is administering the stimulus modality. By way of further example, if the user (e.g., user 138) applies the stimulus modality, the reactivity system display (e.g., display 130) may display a graphical user interface as described below with regard to FIGS. 5-10.

The method may also include, in parallel with the predetermined sequence, receiving, via the at least one processor (e.g., user interface 132) of the reactivity system (e.g., system 120), stimulus data that includes EEG data captured by sensors (e.g., sensors 124) applied to the patient (e.g., patient 136) (Step 204). For example, the sensors (e.g., sensors 124) that capture the stimulus data may be applied to the patient's head, chest, etc.

The stimulus data that includes EEG data may include at least one of:
p a. average voltage level;
b. root mean square (rms) voltage level and/or a peak voltage level;
c. derivatives involving fast Fourier transform (FFT) of recorded brain activity, including spectrogram, spectral edge, peak values, phase spectrogram, power, and/or power ratio (also including variations of calculated power such as average power level, rms power level, and/or a peak power level);
d. measures derived from spectral analysis such as power spectrum analysis, bispectrum analysis, density, coherence, signal correlation, and/or convolution;
e. measures derived from signal modeling such as linear predictive modeling and/or autoregressive modeling;
f. integrated amplitude;
g. peak envelope or amplitude peak envelope;
h. periodic evolution;
i. suppression ratio;
j. coherence and phase delays;
k. wavelet transform of recorded electrical signals, including spectrogram, spectral edge, peak values, phase spectrogram, power, and/or power ratio of measured brain activity;
l. wavelet atoms;
m. bispectrum, autocorrelation, cross bispectrum, and/or cross correlation analysis;
n. data derived from a neural network, a recursive neural network, and/or deep learning techniques;
o. identification of the recording element(s) detecting local minimum and/or maximum of parameters derived from elements a-n; and/or
p. evaluation of symmetry of parameters derived from elements a-n.

Additionally, the method may further include, in parallel with the predetermined sequence, receiving sensor data from at least one sensor (e.g., sensor(s) 126) configured to capture one or more of a biological property or a physiological property of the patient (e.g., patient 136) different than the EEG data.

The one or more physiological property may include at least one of: pupillary diameter, heart rate, heart rate variability, respiratory rate, respiratory variability, tidal volume, airway pressure, oxygen saturation, arterial blood pressure, venous blood pressure, temperature, skin conductance, serum chemistry, serum metabolites, CSF chemistry, CSF metabolites, and/or brain oxygen extraction.

The sensor data may be captured from: a bedside physiological monitor, a bedside computer that processes physiological data, data stored in an electronic medical record, or direct input from clinical personnel.

The method may further include forming the reactivity profile by, via the at least one processor (e.g., processor 134) of the reactivity system (e.g., system 120), temporally aligning the received stimulus data with the predetermined sequence, the aligning including defining segments of the received stimulus data based on temporal positions of the plurality of reactivity outputs in the predetermined sequence (Step 206).

The reactivity profile may indicate the patient's (e.g., patient 136) real-time reactions to the predetermined sequence, allowing for a user (e.g., user 138) to easily identify the patient's responsiveness to the stimulus modalities. More specifically, the temporal alignment of the received stimulus data with the predetermined sequence may be usable to generate a visual depiction of the patient reaction in parallel to the events of the predetermined sequence. Furthermore, the reactivity profile may include defined segments of the received stimulus data, which may be defined based on the temporal positions of the plurality of reactivity outputs. For example, the received stimulus data may include defined segments bounded by the start times and the end times of the plurality of reactivity outputs (e.g., the at least one stimulus period and/or at least one washout period of the predetermined sequence), where the defined segments also include the stimulus data within such start times and end times. The defined segments may allow for a user (e.g., user 138) to look at the stimulus data to evaluate the received stimulus data in response to one of the stimulus periods or washout periods of the predetermined sequence. Furthermore, in some embodiments, the aligning may be done automatically, which may, for example, reduce the risk of user error.

The reactivity profile may also include defined segments of the

temporally aligned received sensor data. The defined segments of the received sensor data may include defining the start times and the end times of the sensor data, where the start times and the end times are associated with the plurality of reactivity outputs. For example, a defined segment may include the start time and the end time of a stimulus period, as well as the sensor data within such start time and end time. Additionally, for example, a defined segment may include the start time and the end time of a washout period, as well as the sensor data within such start time and end time.

Aligning the received sensor data with the predetermined sequence may allow the reactivity system (e.g., system 120) and/or the user (e.g., user 138) to perform further analysis regarding the patient's reaction, as illustrated by the sensor data, to the plurality of reactivity outputs in the predetermined sequence. For example, the defined segments allow for the reactivity system to isolate some or all of the sensor data based on when some or all of the plurality of reactivity outputs started or ended.

The aligning of the received stimulus data with the predetermined sequence and the aligning of the received sensor data may occur in parallel to each other. For example, the reactivity system 120 may align both the stimulus data and the received sensor data with the predetermined sequence. This may thus result in the alignment of the defined segments because the start time and end time of the reactivity outputs are the same for both the received stimulus data and the received sensor data. This may thus provide a more detailed view of the patient's (e.g., patient 136) reaction to the plurality of reactivity outputs relative to conventional techniques because the user (e.g., user 138) is able to consider the sensor data in conjunction with the stimulus data.

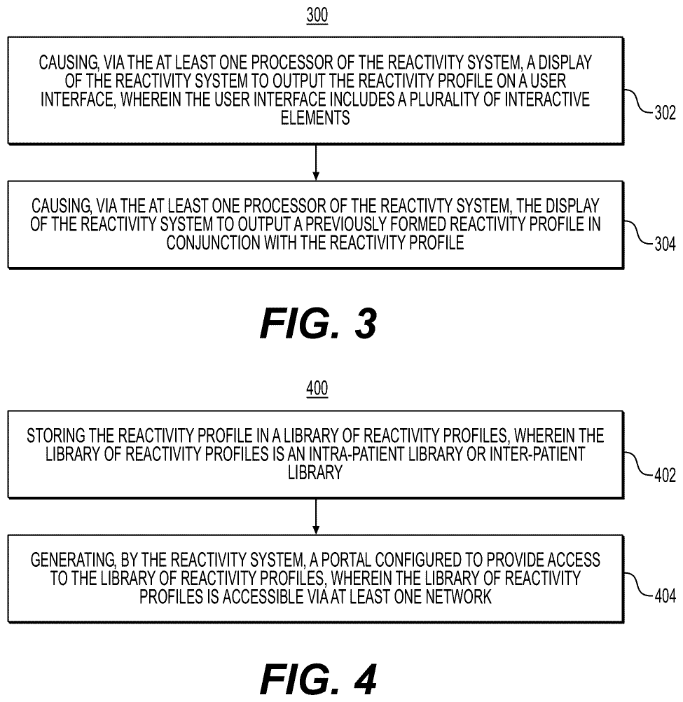

FIG. 3 depicts an exemplary method 300 for displaying a reactivity profile of a patient (e.g., patient 136), according to one or more embodiments.

The method may include causing, via the at least one processor (e.g., processor 132) of the reactivity system (e.g., system 120), a display (e.g., display 130) of the reactivity system to output the reactivity profile on a user interface (e.g., user interface 132), wherein the user interface includes a plurality of interactive elements (Step 302). The display may be similar to the display that indicates the commands to a user. The display may also be separate from the display that indicates the commands to the user. For example, the display may be a remote display (e.g., of remote system 142), such as a mobile device, that allows a neurologist to view the reactivity profile remotely.

Additionally, the display (e.g., display 130) may allow the isolation of some or all of the defining segments. For example, a user (e.g., user 138) may input commands to isolate the defining segments of both the stimulus data and sensor data that correspond to a particular stimulus period. A user would then be able to perform an efficient analysis of such defining segments. Further, the display may allow a user to identify and label a specific segment as “reactive” or “non-reactive.”

Moreover, the reactivity system (e.g., system 120) may automatically isolate a subset of defining segments, which may be output on the display (e.g., display 130). For example, the reactivity system may automatically select a certain number of defining segments that include the highest stimulus data and/or sensor data, as well as a certain number of defining segments that include the lowest stimulus data and/or sensor data. The reactivity system 120 may have a particular threshold, which when crossed, would indicate a severe condition of the patient. The reactivity system may output the defining segments of the reactivity profile that correspond to surpassing such threshold.

The method may further include causing, via the at least one processor (e.g., processor 132) of the reactivity system (e.g., system 120), the display (e.g., display 130) of the reactivity system to output a previously formed reactivity profile in conjunction with the reactivity profile (Step 304). For example, the previously formed reactivity profile may correspond to the current patient. Additionally, the display may automatically indicate areas where there are similar results and/or vastly different results when the reactivity profile and the previously formed reactivity profile are compared. For example, the reactivity system may visually depict segments with similar and/or different results in such a manner that they may be visually compared, e.g., in alignment with each other, overlapping, etc. The visual comparison may utilize numerical or graphical representations of the compared reactivity profiles. The visual comparison may also include a concurrent display of physiological variables that could influence the results of a particular reactivity profile (e.g., the presence of sedating medications, conditions of low blood pressure or high intracranial pressure, etc.). The reactivity system may allow for a user (e.g., user 18) to input a particular range, where results that are within the range, or are outside of the range, are highlighted for the user's attention.

FIG. 4 depicts an exemplary method 400 for generating a portal for providing access to a library of reactivity profiles of a patient (e.g., patient 136), according to one or more embodiments.

The reactivity profile may be stored in a library of reactivity profiles, e.g., in a remote system (e.g., remote system 142) (Step 402). For example, the library may include one or more of an intra-patient library and/or an inter-patient library. The intra-patient library may include multiple reactivity profiles that correspond to the same patient, where the reactivity profiles correspond to EEG reactivity tests that were administered at different previous times. The inter-patient library may include multiple reactivity profiles for different patients. The inter-patient library may also include multiple reactivity profiles for the same patient, in addition to reactivity profiles for different patients. The library may or may not be limited to a particular hospital, hospital network, clinic, clinic network, physician, and/or clinician.

The method may further include generating, by the reactivity system (e.g., system 120) or the remote system (e.g., remote system 142), a portal configured to provide access to the library of reactivity profiles, where the library of reactivity profiles may be accessible via at least one network (e.g., electric network 114) (Step 404). The reactivity system may automatically display the portal on a system display (e.g., a display of a client device used to access the portal), on request and/or as soon as the reactivity profile has been formed. The portal may prompt the physician 102 (e.g., user 138) or the like to enter credentials before accessing the reactivity profile. Upon entering credentials, the library of reactivity profiles may be displayed to the physician 102 (e.g., user 138). Additionally, the credentials of the physician 102 (e.g., user 138) may only allow the physician 102 (e.g., user 138) to view a subset of the reactivity profiles, where the subset corresponds to profiles that meet the physician 102's (e.g., user 138) credentials.

Additionally, the portal may allow a physician 102 (e.g., user 138) to remotely access the library of reactivity profiles, in order to perform additional analysis of the reactivity profile. A physician 102 (e.g., user 138) may log into at least one network (e.g., electric network 114), provide credentials and/or follow an authentication process, and then provide credentials to the portal, in order to access the library of reactivity profiles. The portal may allow the physician 102 (e.g., user 138) to make additional annotations in the reactivity profile. Such annotations may include flagging areas of abnormal results, diagnoses, recommendations for next steps for the patient (e.g., patient 136), and/or tagging another physician and/or clinician to view the reactivity profile.

FIGS. 5-10 illustrate an exemplary graphical user interface (e.g., user interface 132) for administering a standardized EEG reactivity test to a patient (e.g., patient 136), according to one or more embodiments.

FIG. 5 depicts a “Prepare for Reactivity Test” page of the exemplary graphical user interface (e.g., user interface 132), according to one or more embodiments. In order to effectively evaluate a patient's reactivity to stimulation, the patient (e.g., patient 136) should be off any medications that could otherwise minimize brain activity. As a result, prior to the performance of EEG reactivity testing, the user should stop the administration of medications for a period of time to “wash-out” such medications from the patient's system. Example medications may include at least one of: sedating medications, analgesic medications, paralytic medications, anti-epileptic medications, and/or other medications affecting neuronal function, such as lidocaine, stimulants, and/or lithium. The sedating medications may include at least one of: inhaled anesthetics, intravenous anesthetics, benzodiazepines, barbiturates, anxiolytics, opioids, and/or drugs of abuse.

The “Prepare for Reactivity Test” page asks the user (e.g., user 138) to confirm that the patient (e.g., patient 136) is off of medications that may interfere with the patient's EEG reactivity. The user may also input how long the patient has been off of sedation. This provides for standardization of the EEG reactivity test because the amount of time the patient has been off of interfering medications has been quantified and recorded. A user reviewing the test results may notice a correlation between the length of time a patient has been off of interfering medications and the EEG reactivity test data. Additionally, the page may also require the user to input a period of time that the patient has been off of sedation. If the user does not input a period of time, the system may not allow the user to move forward with the testing.

FIG. 6 depicts a “Stimulation Parameters” page of the exemplary graphical user interface (e.g., user interface 132), according to one or more embodiments. The “Stimulation Parameters” page specifies the types of stimulus modalities that the user (e.g., user 138) may administer to the patient (e.g., patient 136). For example, the stimulus modalities may include bilateral (manual) eye opening, loud verbal stimulation (call the patient's name, ask them to move, etc.), or vigorous sternal rub. The “Simulation Parameters” page may also allow the user to independently note or record the nature of the stimulus performed, if the performed stimulus is different than the specified stimulus.

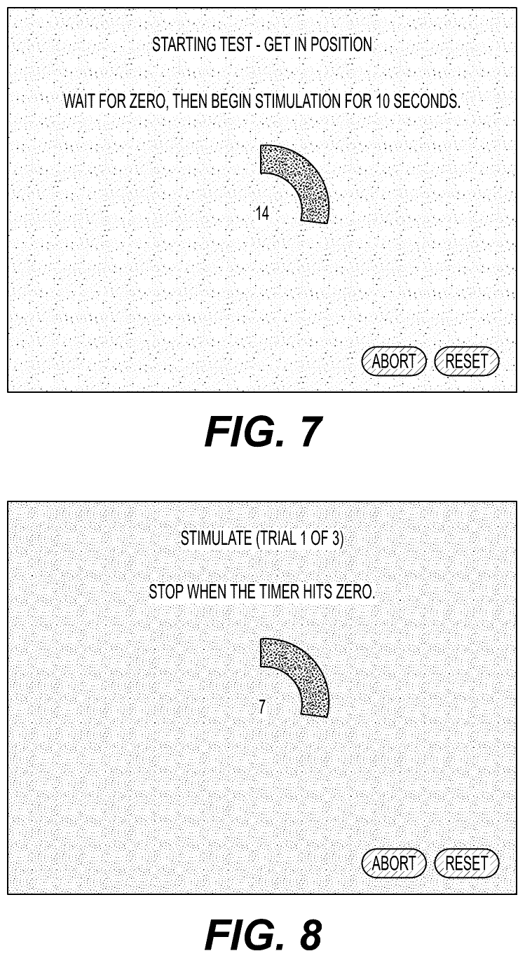

FIG. 7 depicts a “Starting Test” page of the exemplary graphical user interface (e.g., user interface 132), according to one or more embodiments. The “Starting Test” page displays a countdown timer, which allows the user to get into position to administer a stimulation modality on the patient. However, the user may not administer any stimulation during this time. In some embodiments, the “Starting Test” page may include a depiction, animation, guide, or the like regarding the position for the user and/or the stimulus to be performed.

FIG. 8 depicts a “Stimulate” page of the exemplary graphical user interface (e.g., user interface 132), according to one or more embodiments. The “Stimulate” page may display a countdown timer of a stimulus period that corresponds to a predetermined physiological stimulus period. In some embodiments, the “Stimulate” page may include a depiction, animation, guide, or the like regarding the position for the user (e.g., user 138) and/or the stimulus to be performed. During this time, the user may administer one of the targeted stimulus modalities to the patient (e.g., patient 136). The page may also display the specific trial number of a total number of stimulus trials of the predetermined sequence. For example, in FIG. 8, there are three stimulus trials of the predetermined sequence.

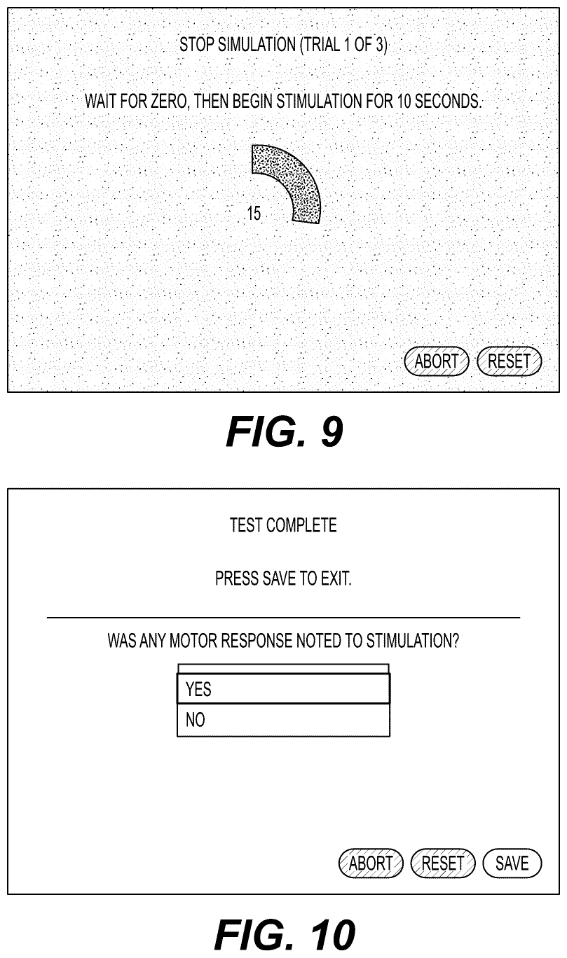

FIG. 9 depicts a “Stop Stimulation” page of the exemplary graphical user interface (e.g., user interface 132), according to one or more embodiments. The “Stop Stimulation” page may display a countdown timer, e.g., that may correspond to the washout period that is associated with a predetermined post-stimulus settling period. During this time, the user (e.g., user 138) may refrain from administering any of the stimulus modalities to the patient (e.g., patient 136). This time period allows for the patient's neurological functions to reset. The page may also display the number of total trials (washout periods) of the predetermined sequence. For example, in FIG. 8, there are three trials (washout periods) of the predetermined sequence.

FIG. 10 depicts a “Test Complete” page of the exemplary graphical user interface (e.g., user interface 132), according to one or more embodiments. The “Test Complete” page may display an opportunity for the user (e.g., user 138) to note whether the patient (e.g., patient 136) had a motor or otherwise observed physiological response to the previously administered stimulation modality.

The steps corresponding to FIGS. 8-10 may repeat according to the predetermined sequence. For example, if there are three sets of stimulation periods and three sets of washout periods, FIGS. 8-10 may repeat three times.

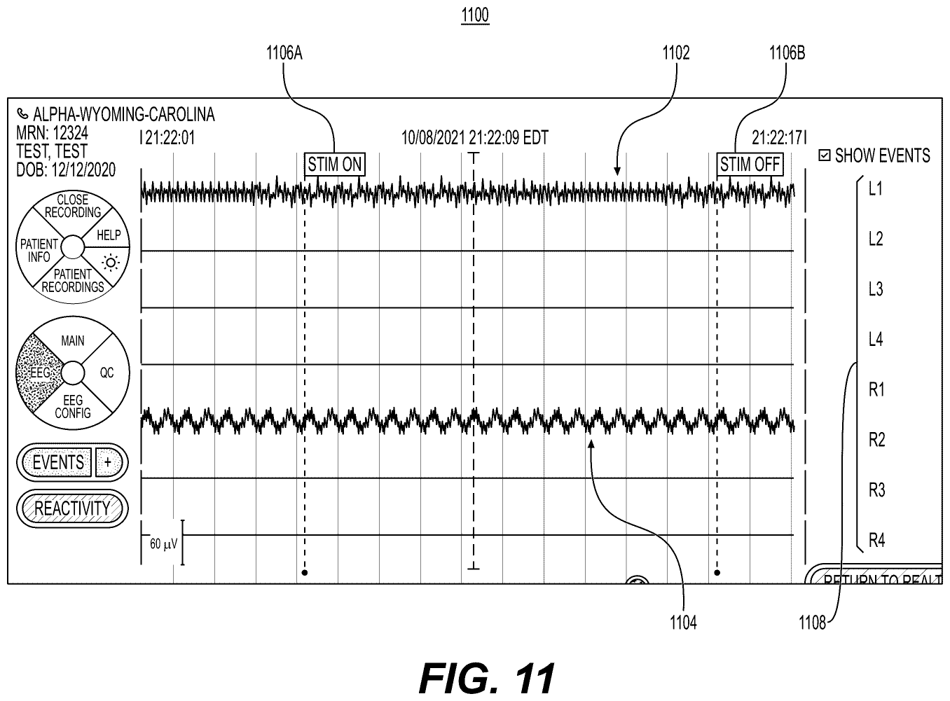

FIG. 11 depicts a reactivity profile interface 1100 of aligned received stimulus data of an exemplary graphical user interface (e.g., user interface 132), according to one or more embodiments.

The reactivity profile interface 1100 may include received stimulus data 1102 and 1104, annotations 1106A and 1106B, and EEG channel identifiers 1108 (in this case representing four independently recorded EEG channels from each of the left brain (L1-L4) and right brain (R1-R4) hemispheres). The received stimulus data 1102 may indicate one channel of raw EEG waveform data (in this embodiment, the first channel on the left side). The received stimulus data 1104 may indicate another channel of raw EEG waveform data (in this case, the first channel on the right side). The received stimulus data 1102 and 1104 may be aligned with the predetermined sequence, where the reactivity profile interface 1100 indicates where the stimulus begins 1106A and where the stimulus ends 1106B. Furthermore, the reactivity profile interface 1100 may include a defined segment of received stimulus data 1102 and 1104, where the defined segment is bounded by the beginning of the stimulation 1106A and the end of the stimulation 1106B.

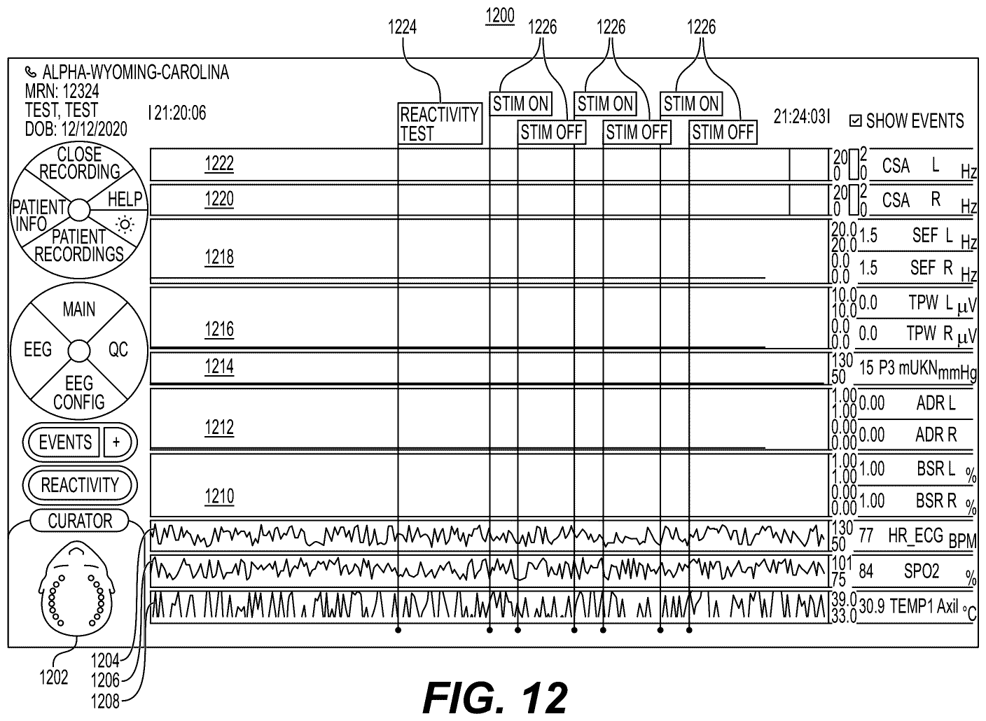

FIG. 12 depicts a main reactivity profile interface 1200 of the aligned received stimulus data and received sensor data of an exemplary graphical user interface (e.g., user interface 132), according to one or more embodiments.

An additional reactivity profile interface 1200 may include EEG electrode connection or recording quality status 1202, received sensor data 1204-1208 and 1214, received stimulus data 1210-1212 and 1216-1222, reactivity test start indicator 1224, and defined segments 1226 of the received sensor data 1204-1208 and 1214 and received stimulus data 1210-1212 and 1216-1222. The EEG electrode status 1202 may indicate if all of the scalp electrodes are working (e.g., indicated by a green color) or if one or more of the scalp electrodes are offline (e.g., indicated by a red color). The received sensor data 1204-1208 and 1214 may indicate elevated heart rate (HR), decreased oxygen saturation (SP02), patient temperature (TEMP1 Axil), any other physiological variable (P3 mUKN), other recorded physiological data, medication data, laboratory data, or other clinical information as recorded by a clinician. The received processed EEG data may include Burst Suppression Ratio (BSR) 1210, Alpha Delta Ratio (ADR) 1212, Total Power (TPW) 1216, Spectral Edge Frequency (SEF) 1218, Compressed Spectral Array on the right side (CSA R) 1220, Compressed Spectral Array on the left side (CSA L) 1222, and/or other processed EEG derivatives. The stimulus labels 1226 may indicate where each of the stimuli began and ended.

The received sensor data 1204-1208 and 1214 and the received stimulus data 1210-1212 and 1216-1222 may be aligned, so that such data has synchronized temporal positions. Additionally, the aligned received sensor data 1204-1208 and 1214 and the received stimulus data 1210-1212 and 1216-1222 may be defined in segments, where the segments are bounded by the stimulus labels 1226. For example, each of the stimulus labels 1226 (e.g., “stim on” and “stim off”) may indicate where a defined segment begins and ends.

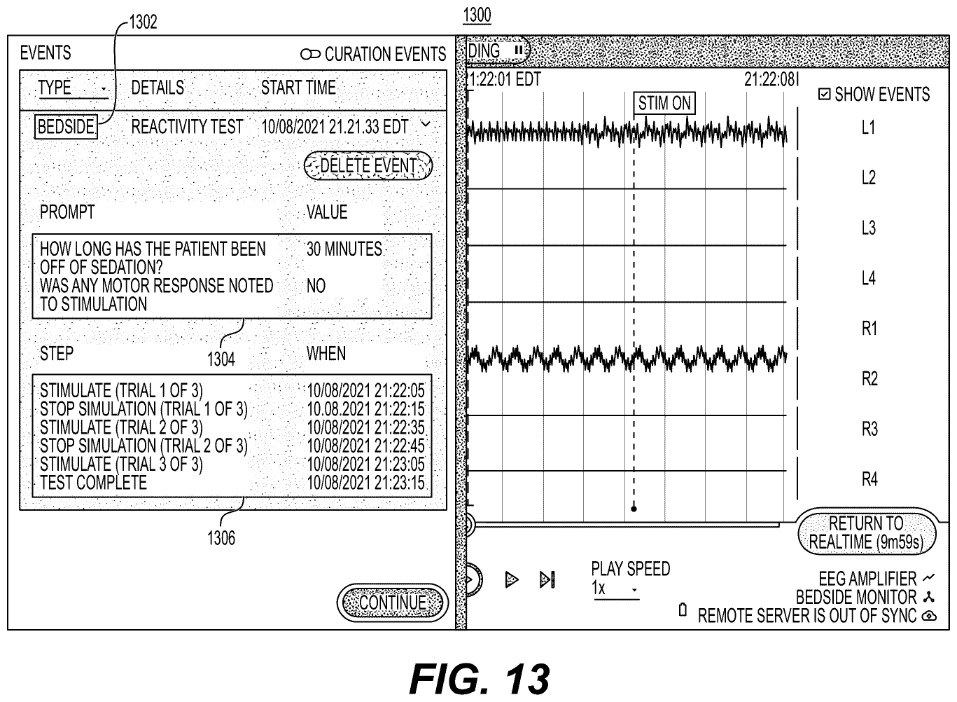

FIG. 13 depicts an overview interface 1300 of an exemplary graphical user interface (e.g., user interface 132), according to one or more embodiments. The overview interface 1300 may provide a log of the EEG reactivity test data. For example, the overview interface 1300 may include the location of the test (e.g., bedside 1302), details regarding how long the patient was off of sedation and/or whether any motor responses to the stimulation were noted (e.g., the patient was off sedation for 30 minutes and no motor responses were noted 1304), and/or timestamps indicating the beginning and the end of each stimulation 1306.

It should be understood that embodiments in this disclosure are exemplary only, and that other embodiments may include various combinations of features from other embodiments, as well as additional or fewer features.

In general, any process or operation discussed in this disclosure that is understood to be computer-implementable, such as the processes illustrated in FIGS. 2-4, may be performed by one or more processors of a computer system, such any of the systems or devices in the environment 100 of FIG. 1A, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices in FIG. 1A. One or more processors of a computer system may be included in a single computing device or distributed among a plurality of computing devices. A memory of the computer system may include the respective memory of each computing device of the plurality of computing devices.

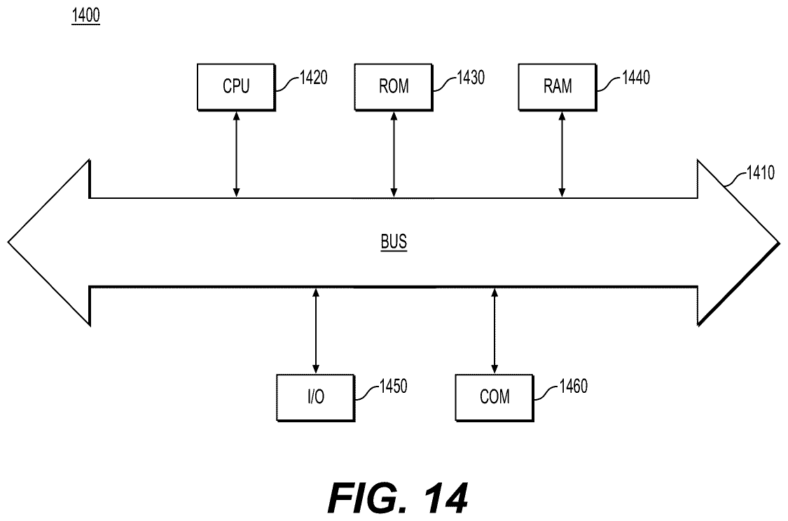

FIG. 14 depicts a simplified functional block diagram of a computer 900 that may be configured as a device for executing the methods of FIGS. 2-4, according to exemplary embodiments of the present disclosure. For example, device 1400 may include a central processing unit (CPU) 1420. CPU 1420 may be any type of processor device including, for example, any type of special purpose or a general-purpose microprocessor device. As will be appreciated by persons skilled in the relevant art, CPU 1420 also may be a single processor in a multi-core/multiprocessor system, such system operating alone, or in a cluster of computing devices operating in a cluster or server farm. CPU 1420 may be connected to a data communication infrastructure 1410, for example, a bus, message queue, network, or multi-core message-passing scheme.

Device 1400 also may include a main memory 1440, for example, random access memory (RAM), and also may include a secondary memory 1430. Secondary memory 1430, e.g., a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage unit may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 1430 may include other similar means for allowing computer programs or other instructions to be loaded into device 1400. Examples of such means may include a program cartridge and cartridge interface (such as that found in video game devices), a removable memory chip (such as an EPROM, or PROM) and associated socket, and other removable storage units and interfaces, which allow software and data to be transferred from a removable storage unit to device 1400.

Device 1400 also may include a communications interface (“COM”) 1460. Communications interface 1460 allows software and data to be transferred between device 1400 and external devices. Communications interface 1460 may include a modem, a network interface (such as an Ethernet card), a communications port, a PCMCIA slot and card, or the like. Software and data transferred via communications interface 1460 may be in the form of signals, which may be electronic, electromagnetic, optical, or other signals capable of being received by communications interface 1460. These signals may be provided to communications interface 1460 via a communications path of device 1400, which may be implemented using, for example, wire or cable, fiber optics, a phone line, a cellular phone link, an RF link or other communications channels.

The hardware elements, operating systems and programming languages of such equipment are conventional in nature, and it is presumed that those skilled in the art are adequately familiar therewith. Device 1400 also may include input and output ports 1450 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed embodiments may be applicable to any environment, such as a desktop or laptop computer, an automobile entertainment system, a home entertainment system, etc. Also, the disclosed embodiments may be applicable to any type of Internet protocol.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for generating a reactivity profile of a patient, the method comprising:
causing, via at least one processor of a reactivity system, a reactivity device to generate a plurality of reactivity outputs according to a predetermined sequence, the plurality of reactivity outputs respectively associated with different stimulus modalities;
in parallel with the predetermined sequence, receiving, via the at least one processor of the reactivity system, stimulus data that includes electroencephalogram (EEG) data captured by sensors applied to the patient; and
forming the reactivity profile by, via the at least one processor of the reactivity system, temporally aligning the received stimulus data with the predetermined sequence, the aligning including defining segments of the received stimulus data based on temporal positions of the plurality of reactivity outputs in the predetermined sequence.

2. The computer-implemented method of claim 1, further comprising:
causing, via the at least one processor of the reactivity system, a display of the reactivity system to output the reactivity profile on a user interface, wherein the user interface includes a plurality of interactive elements.

3. The computer-implemented method of claim 2, further comprising:
causing, via the at least one processor of the reactivity system, the display of the reactivity system to output a previously formed reactivity profile in conjunction with the reactivity profile.

4. The computer-implemented method of claim 1, wherein the different stimulus modalities include at least two of: at least one visual modality, at least one auditory modality, and at least one somatosensory modality.

5. The computer-implemented method of claim 1, wherein each of the plurality of reactivity outputs respectively includes:
at least one washout period with a length based on a predetermined post-stimulus settling period; and
at least one stimulus period with a stimulus length based on a predetermined physiological stimulus period, the at least one stimulus period preceding the at least one washout period.

6. The computer-implemented method of claim 1, further comprising:
in parallel with the predetermined sequence, receiving, via the at least one processor of the reactivity system, sensor data from at least one sensor configured to capture one or more of a biological property or a physiological property of the patient different than the EEG data; and
temporally aligning the received sensor data with the predetermined sequence, the aligning including defining segments of the received sensor data based on temporal positions of the plurality of reactivity outputs in the predetermined sequence.

7. The computer-implemented method of claim 1, wherein the defining segments of the received stimulus data includes defining start times and end times for stimuli associated with the plurality of reactivity outputs.

8. The computer-implemented method of claim 1, further comprising:
storing the reactivity profile in a library of reactivity profiles, wherein the library of reactivity profiles includes one or more of an intra-patient library or an inter-patient library.

9. The computer-implemented method of claim 8, further comprising:
generating, by the reactivity system, a portal configured to provide access to the library of reactivity profiles, wherein the library of reactivity profiles is accessible via at least one network.

10. A computer system for generating a reactivity profile of a patient, the computer system comprising:
a reactivity device operable to generate a reactivity output;
a plurality of sensors configured to be applied to the patient;
at least one memory storing processor-readable instructions; and
at least one processor operatively connected to the reactivity device, the plurality of sensors, and the at least one memory, the at least one processor configured to execute the processor-readable instructions to perform a plurality of operations, including:
causing the reactivity device to generate a plurality of reactivity outputs according to a predetermined sequence, the plurality of reactivity outputs respectively associated with different stimulus modalities;
in parallel with the predetermined sequence, receiving, via the reactivity device, stimulus data that includes electroencephalogram (EEG) data captured by the plurality of sensors; and
forming the reactivity profile by temporally aligning the received stimulus data with the predetermined sequence, the aligning including defining segments of the received stimulus data based on temporal positions of the plurality of reactivity outputs in the predetermined sequence.

11. The computer system of claim 10, further comprising:
a display;
wherein the plurality of operations further include causing the display to output the reactivity profile on a user interface, wherein the user interface includes a plurality of interactive elements.

12. The computer system of claim 11, wherein the plurality of operations further include:
causing, via the reactivity system, the display of the reactivity system to output a previously formed reactivity profile in conjunction with the reactivity profile.

13. The computer system of claim 10, wherein the different stimulus modalities include at least two of: at least one visual modality, at least one auditory modality, and at least one somatosensory modality.

14. The computer system of claim 10, wherein each of the plurality of reactivity outputs respectively includes:
at least one washout period with a length based on a predetermined post-stimulus settling period; and
at least one stimulus period with a stimulus length based on a predetermined physiological stimulus period, the at least one stimulus period preceding the at least one washout period.

15. The computer system of claim 10, further comprising:
in parallel with the predetermined sequence, receiving, via the reactivity system, sensor data from at least one sensor configured to capture one or more of a biological property or a physiological property of the patient different than the EEG data; and
temporally aligning the received sensor data with the predetermined sequence, the aligning including defining segments to the received sensor data based on temporal positions of the plurality of reactivity outputs in the predetermined sequence.

16. The computer system of claim 10, wherein the defining segments of the received stimulus data includes defining start times and end times for stimuli associated with the plurality of reactivity outputs.

17. A non-transitory computer-readable medium comprising instructions that are executable by a processor to perform operations for generating a reactivity profile of a patient, the operations including:
causing, via a reactivity system, a reactivity device to generate a plurality of reactivity outputs according to a predetermined sequence, the plurality of reactivity outputs respectively associated with different stimulus modalities;
in parallel with the predetermined sequence, receiving, via the reactivity system, stimulus data that includes electroencephalogram (EEG) data captured by sensors applied to a patient; and
forming the reactivity profile by, via the reactivity system, temporally aligning the received stimulus data with the predetermined sequence, the aligning including defining segments of the received stimulus data based on temporal positions of the plurality of reactivity outputs in the predetermined sequence.

18. The non-transitory computer-readable medium of claim 17, wherein the defining segments of the received stimulus data includes defining start times and end times for stimuli associated with the plurality of reactivity outputs.

19. The non-transitory computer-readable medium of claim 17, further comprising:
storing the reactivity profile in a library of reactivity profiles, wherein the library of reactivity profiles includes at least one of an intra-patient library or an inter-patient library.

20. The non-transitory computer-readable medium of claim 19, further comprising:
generating, by the reactivity system, a portal configured to provide access to the library of reactivity profiles, wherein the library of reactivity profiles is accessible via at least one network.
